(19) United States
(12) Patent Application Publication
(10) Pub. No.: US 2026/0315209 A1
(43) Pub. Date: Oct. 15, 2026

(54) SUBDERMAL HAND IMPLANT APPARATUS AND METHOD

(71) Applicant: Frances Champe Granger, Richmond, VA (US)

(72) Inventor: Frances Champe Granger, Richmond, VA (US)

(21) Appl. No.: 19/429,319

(22) Filed: Dec. 22, 2025

Related U.S. Application Data
(60) Provisional application No. 63/787,825, filed on Apr. 12, 2025.

(51) Int. Cl. A61F 2/12; A61B 17/02; A61F 2/44; A61F 2/46

(52) U.S. Cl. CPC A61F 2/12; A61B 17/025; A61B 17/0293; A61F 2/447; A61F 2/4611

(57) ABSTRACT

A method is disclosed. The method includes making an incision in a dorsal area of a hand, creating a pocket at the incision in the dorsal area by separating a subcutis and a fascia of the hand, and inserting a subdermal hand implant in the pocket. The subdermal hand implant includes a sheet of malleable material.

### CROSS-REFERENCE TO RELATED APPLICATIONS

This application claims priority to U.S. provisional patent application No. 63/787,825 filed Apr. 12, 2025, the entire disclosure of which is incorporated herein by reference.

### TECHNICAL FIELD

The present disclosure generally relates to an implant apparatus and method, and more particularly to a subdermal hand implant apparatus and method.

### BACKGROUND

As people age, they can develop lines and wrinkles on their face, hands, and other parts of the body. While many cosmetic solutions exist for the face, few solutions exist for the hands. Conventional cosmetic techniques for treating the hands typically focus on skin color and age spots. The layer of fat beneath the skin, which provides cushioning and plumpness, diminishes with age. This loss makes the skin appear thinner and allows underlying structures like veins and tendons to become more prominent. Collagen and elastin are proteins that provide skin with strength, elasticity, and the ability to return to its original shape. As we age, the production of these proteins slows down, leading to thinner, less elastic skin that is more prone to wrinkling and sagging. Collagen inserts are typically used in cosmetic procedures to enhance facial features, reduce wrinkles, and improve skin texture.

Fillers can be used on the hands, but tend to be generally ineffective. Conventional cosmetic hand treatments typically have limited duration times for effectiveness. For example, fillers may be used to treat hands cosmetically, but such fillers are not effective for long periods of time and can dissipate into the body, after which no benefit is provided.

Even if conventional techniques such as fillers last a few months, these techniques do not address the bony, veiny look of older hands. Accordingly, a need in the art exists for effective cosmetic treatment of the hands and/or treatment of injured hands.

One attempt at addressing the deficiencies in current techniques for addressing wrinkles is disclosed in U.S. Pat. No. 7,559,952 to Pinchuk (the '952 patent). The '952 patent discloses an elastomeric filament that may be implanted under sagging or wrinkled skin. However, the '952 patent fails to sufficiently address treating the bony, veiny look of older hands.

The exemplary disclosed apparatus and method of the present disclosure are directed to overcoming one or more of the shortcomings set forth above and/or other deficiencies in existing technology.

### SUMMARY OF THE DISCLOSURE

In one exemplary aspect, the present disclosure is directed to a method. The method includes making an incision in a dorsal area of a hand, creating a pocket at the incision in the dorsal area by separating a subcutis and a fascia of the hand, and inserting a subdermal hand implant in the pocket. The subdermal hand implant includes a sheet of malleable material.

In another aspect, the present disclosure is directed to a subdermal hand implant configured to be implanted in a dorsal area of a hand. The subdermal hand implant includes a sheet of malleable material, wherein the sheet of malleable material is a permanent-form implant that is non-dissipating and non-temporary. The sheet of malleable material is configured in a dorsal hand shape. Each of a length dimension and a width dimension of the sheet of malleable material is between 5 times and 50 times greater than a thickness dimension of the sheet of malleable material.

### BRIEF DESCRIPTION OF THE DRAWINGS

FIG. 1A is a side view of an exemplary apparatus of the present invention;

FIG. 1B is a top view of the exemplary apparatus illustrated in FIG. 1A;

FIG. 1C is a sectional view of the exemplary apparatus illustrated in FIG. 1A;

FIG. 1D is another sectional view of the exemplary apparatus illustrated in FIG. 1A;

FIG. 1E is a sectional view of another exemplary apparatus of the present invention;

FIG. 2 is a top view of an exemplary apparatus of the present invention;

FIG. 3 is another top view of the exemplary apparatus illustrated in FIG. 2;

FIG. 4 is a schematic view of an exemplary apparatus of the present invention;

FIG. 5 is another top view of the exemplary apparatus illustrated in FIG. 4;

FIG. 5A is a top view of another exemplary apparatus of the present invention;

FIG. 6 is a top view of another exemplary apparatus of the present invention;

FIG. 7 is a sectional view of the exemplary apparatus illustrated in FIG. 6;

FIG. 8 is a top view of another exemplary apparatus of the present invention;

FIG. 9 is a top view of another exemplary apparatus of the present invention; and

FIG. 10 illustrates an exemplary process of installing and using at least some exemplary embodiments of the present disclosure.

### DETAILED DESCRIPTION AND INDUSTRIAL APPLICABILITY

The exemplary disclosed apparatus and method may provide a subdermal hand implant. The exemplary disclosed subdermal hand implant may be a cosmetic implant for mitigating (e.g., covering) signs of aging or injury. For example, the exemplary disclosed subdermal hand implant may treat wrinkles and/or a bony and/or veiny appearance of hands due to aging.

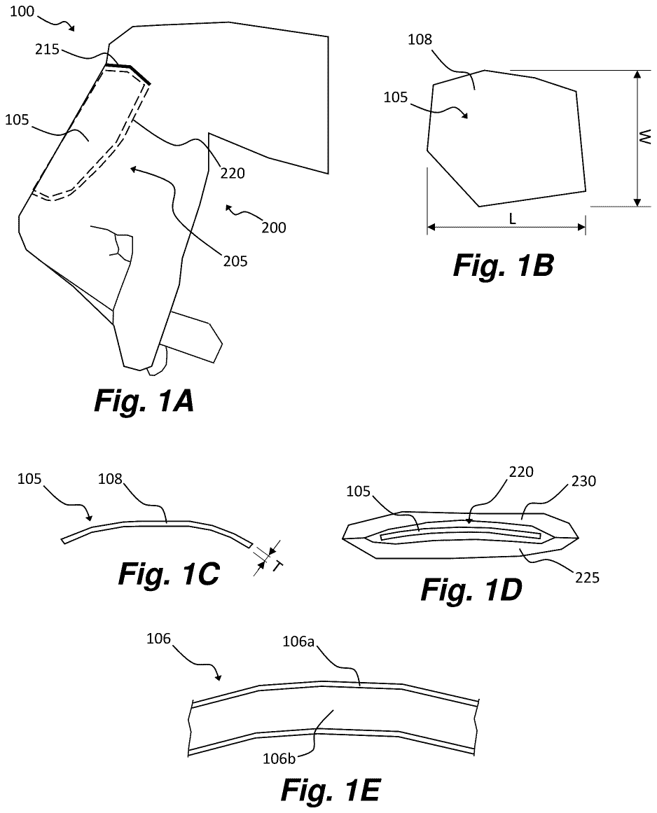

FIGS. 1A, 1B, 1C, 1D, 2, and 3 illustrate a first exemplary embodiment of the exemplary disclosed apparatus. System 100 may be a system for providing a subdermal hand implant. System 100 may include an apparatus such as an implant 105 that may be any suitable implant such as a subdermal implant (e.g., a subdermal hand implant).

Implant 105 may be formed (e.g., manufactured or constructed) from any suitable material for insertion below the skin. The exemplary disclosed subdermal hand implant may be any suitable malleable (e.g., flexible, elastic, and/or malleable) material. Implant 105 may be formed from any suitable soft, flexible material. Implant 105 may be formed from gel such as silicone gel (e.g., cohesive silicone gel). Implant 105 may be formed from silicone, hyaluronic acid, collagen, calcium hydroxyapatite, Poly-L-lactic acid (PLLA), polymethylmethacrylate (PMMA), human fat, and/or any other suitable material for subdermal use. Implant 105 may also be formed from plastic or acrylic material. In at least some exemplary embodiments, implant 105 may be formed from any suitable material that may allow for tissue to grow into the tissue and provide a more natural appearance such as, for example, a porous material such as porous polyethylene or expanded polytetrafluoroethylene. In at least some exemplary embodiments, implant 105 may be formed from any suitable material that may have similar properties to human bone such as, for example, ceramic material (e.g., hydroxyapatite). Implant 105 may be formed to have a solid, flexible form. Also for example, implant 105 may be a malleable (e.g., flexible, elastic, and/or malleable) material that may be encased in a housing. For example, implant 105 may be a solid and/or fluid material that may be housed or encased in a housing (e.g., an encasement). In at least some exemplary embodiments, implant 105 may include a housing such as a shell (e.g., a silicone shell or shell formed from other suitable material for example as disclosed herein) housing a fill (e.g., a saline fill or a cohesive fill). Implant 105 may include a malleable shell housing a fluid fill. For example, the exemplary disclosed shell of implant 105 may be formed from any suitable material that is biocompatible and substantially resistant to tearing (e.g., a synthetic material such as silicone rubber). For example, the exemplary disclosed shell may be a silicone shell formed from polydimethylsiloxane (PDMS) material. The exemplary disclosed filler may be any suitable sterile and non-migrating material (e.g., compatible to avoid leakage or inflammation) for example as described herein. FIG. 1E illustrates an exemplary disclosed implant (e.g., implant 106) having the exemplary disclosed shell (e.g., shell 106a) housing the exemplary disclosed filler (e.g., filler 106b). In at least some exemplary embodiments, implant 105 may be a silicone pouch filled with fat or any other suitable fill (e.g., for example as disclosed herein). In at least some exemplary embodiments, the silicone pouch may be filled with hyaluronic acid or a similar material that may not negatively affect the silicone).

In at least some exemplary embodiments, implant 105 may be a malleable medical-grade P implant that may be formed from silicone material. Implant 105 may be formed, casted, printed (e.g., a silicone object made via 3D-printing), and/or made via any other suitable technique.

As illustrated in FIGS. 1A, 1D, 2, and 3, implant 105 may be implanted at the back of a hand 200 of a user (e.g., patient). For example, implant 105 may be implanted at a dorsal area 205 (e.g., a dorsal surface) of hand 200 of a user.

Implant 105 may be a permanent implant. For example, in contrast to substances such as fillers that may dissipate into the body, implant 105 may be a permanent-form implant that may be non-dissipating and/or non-temporary. Implant 105 may be a removable implant. For example, implant 105 may be a permanent-form implant that may be inserted into a body (e.g., hand 200) of a user and later removed from hand 200.

Implant 105 may have an elongated shape for covering a desired surface portion of dorsal area 205. For example as illustrated in FIGS. 1B and 1C, implant 105 may have a length L and a width W having dimensions that may be greater (e.g., significantly greater) than a dimension of a thickness T of implant 105. For example, length L and width W may be between about 10 mm and about 100 mm, between about 10 mm and about 50 mm, or any other suitable dimensions, while thickness T may be between about 1 mm and about 10 mm, between about 1 mm and about 8 mm, between about 2 mm and about 5 mm, or any other suitable dimensions. Implant 105 may have a relatively constant thickness T that may remain relatively similar across an area of implant 105. A dimension of L and/or W may be between about 2 times and about 50 times greater or more, between about 5 times and about 50 times greater, or between about 10 times and about 40 times greater than a dimension of thickness T. Implant 105 may be of an elongated shape. For example, implant 105 may be a malleable (e.g., flexible, elastic, and/or malleable) sheet or plate. Implant 105 may include or may be a sheet of malleable material (e.g., a sheet 108 as illustrated in FIGS. 1B and 1C).

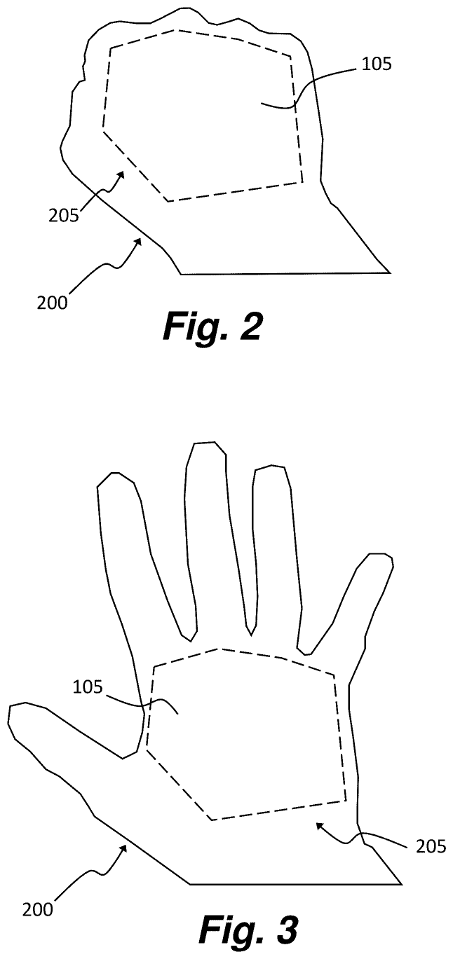

Implant 105 may be shaped or configured to fit dorsal area 205 or a portion of dorsal area 205. For example as illustrated in FIGS. 1A, 2, and 3, implant 105 may be shaped to correspond to a shape of a back of hand 200. Implant 105 may have an asymmetrical shape corresponding to a shape or contours of dorsal area 205. For example, implant 105 may have a dorsal hand shape as illustrated in FIGS. 2 and 3. Implant 105 may be formed in custom sizes or configurations that may be sized to a hand of a particular user or a particular group of users (e.g., different hand sizes for women or men). For example, implant 105 may allow for an entire dorsal area 205 of a user's hand 200 to be treated (e.g., to have a relatively younger appearance).

In at least some exemplary embodiments and as illustrated in FIGS. 1C and 1D, implant 105 may have a contoured shape that may be formed for example to generally correspond to a shape of a wrist area such as dorsal area 205. For example, implant 105 may have contours that form a generally concaved-downward shape that may generally match dorsal area 205 (e.g., a generally curved shape of the top of the wrist at the carpal bones). The carpal bones include eight bones that create the wrist (e.g., the two rows of carpal bones are connected to two bones of the arm: the ulna bone and the radius bone). For example, implant 105 may be concave with dimensions relatively thicker in a middle portion and tapering down in thickness toward the sides or periphery portions of implant 105 (e.g., including tapering down in thickness toward the wrist). Implant 105 may have a malleable form that may generally correspond to a top surface of dorsal area 205 when implant 105 is at an at-rest shape (e.g., when implant 105 is not subjected to forces that may deform implant 105 from its at rest form, for example when substantially no potential energy is stored in implant 105).

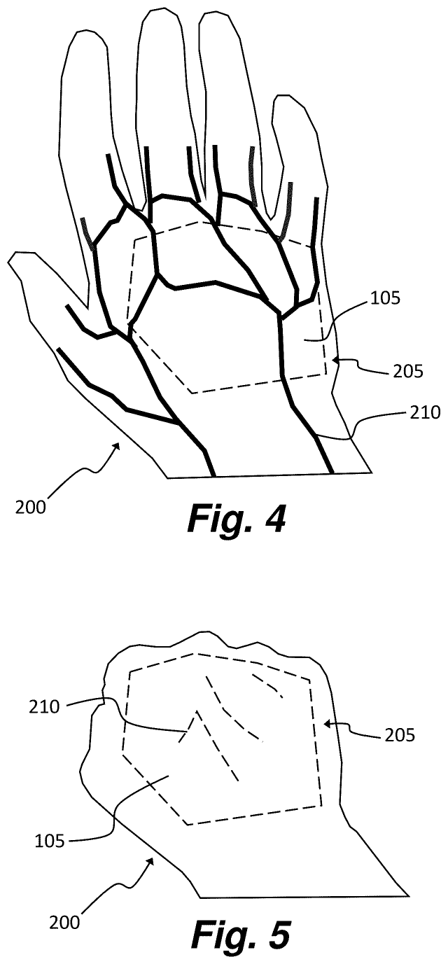

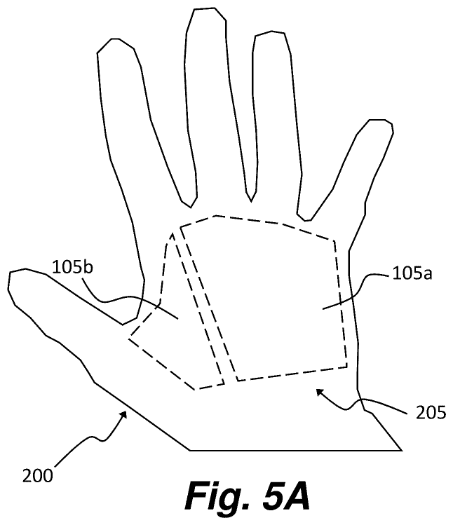

As illustrated in FIGS. 4 and 5, implant 105 may be implanted in an affected area (e.g., implanted directly in an affected area). For example, implant 105 may be implanted directly over an area including one or more visible dorsal veins 210. An effect of implanting implant 105 may be immediate (e.g., immediate or near immediate). For example, implant 105 may immediately (e.g., or nearly immediately) cover (e.g., mitigate an appearance of or hide) bony areas and/or veins such as dorsal veins 210 that may be disposed relatively close to a skin surface of dorsal area 205.

FIG. 5A illustrates another exemplary embodiment of the exemplary disclosed apparatus. Implants 105a and 105b may be generally similar to implant 105 described above. Implants 105a and 105b may be correspondingly shaped to cover (e.g., collectively cover together) dorsal area 205. Implant 105b may be shaped to cover a portion of dorsal area 205 disposed between the index finger and the thumb of a user (e.g., patient). For example, implant 105b may be triangular-shaped, heart-shaped, or include any other suitable polygonal and/or curved shape corresponding to a portion of dorsal area 205 disposed between the user's index finger and thumb for example as illustrated in FIG. 5A. In at least some exemplary embodiments, implant 105b may be implanted without implant 105a (e.g., or alternatively, implant 105a may be implanted without implant 105b).

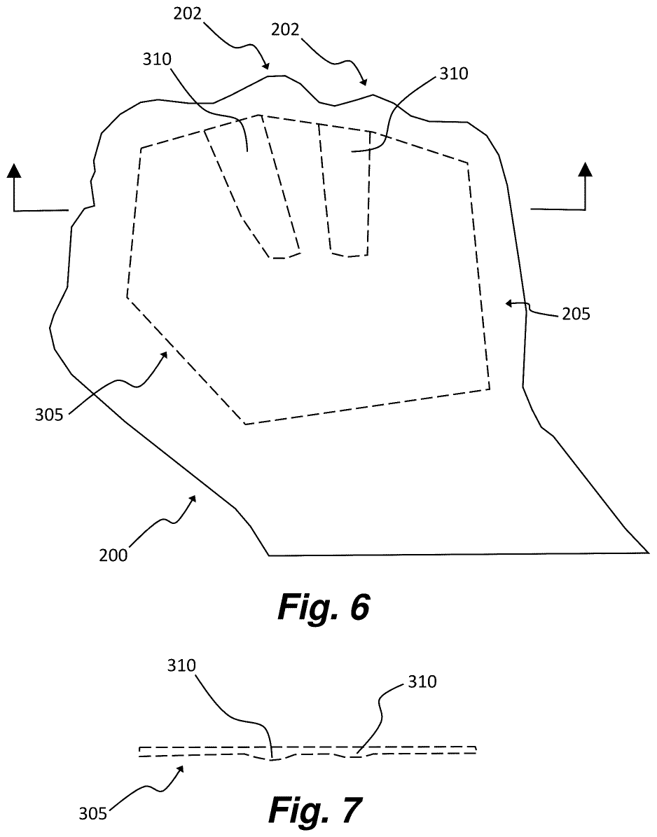

FIGS. 6 and 7 illustrate another exemplary embodiment of the exemplary disclosed apparatus. An implant 305 may be generally similar to implant 105 described above. Implant 305 may include one or more thick portions 310. Thick portion 310 may be a portion of implant 305 having a thickness that may be greater than thickness (e.g., similar to thickness T) of a remainder of implant 305. For example, thick portions 310 may be locations of increased thickness of implant 305 that may be relatively thicker than the remainder of implant 305. For example, thick portion 310 may be up to about 2 or 3 times thicker (e.g., or more) than the thickness of the remainder of implant 305 (e.g., that may be similar to values of thickness T of implant 105 described above). In at least some exemplary embodiments, one or more thick portions 310 may be configured on implant 305 to be disposed between knuckles 202 of hand 200 and/or in any other suitable location so as to reduce or mitigate a bony appearance of dorsal area 205. Thick portions 310 may also be located at positions of dorsal area 205 that may be particularly veiny and/or may have particularly unsightly features.

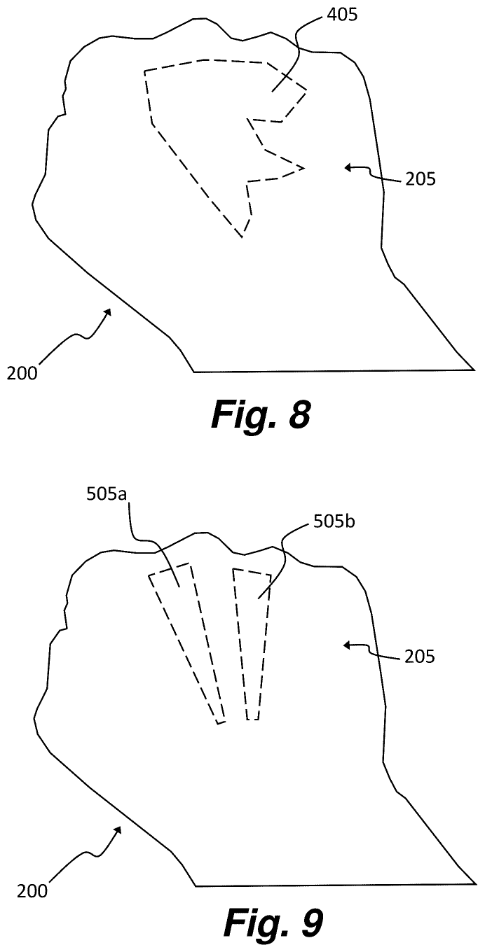

FIG. 8 illustrates another exemplary embodiment of the exemplary disclosed apparatus. An implant 405 may be generally similar to implant 105 described above. Implant 405 may be configured to treat a desired portion of dorsal area 205. For example, implant 405 may be configured to cover typical areas of visible dorsal veins 210 and/or bony locations, or may be configured to cover areas of visible dorsal veins 210 and/or bony locations of hand 200 of a particular user (e.g., patient). Implant 405 may thereby provide for a smaller implant that may effectively treat desired portions of dorsal area 205.

FIG. 9 illustrates another exemplary embodiment of the exemplary disclosed apparatus. A plurality of implants such as implants 505a and 505b (e.g., or any other desired number of implants) may be provided. Implants 505a and 505b may be generally similar to implant 105 described above. Implants 505a and 505b may be configured to treat desired portions of dorsal area 205. Implants 505a and 505b may be a plurality of separate sheets of malleable material for example as described above regarding implant 105. For example, implants 505a and 505b may be configured to cover typical areas of visible dorsal veins 210 and/or bony locations, or may be configured to cover areas of visible dorsal veins 210 and/or bony locations of hand 200 of a particular user (e.g., patient). Implants 505a and 505b may thereby provide for a smaller amount (e.g., surface area) of implants that may effectively treat desired portions of dorsal area 205 at locations that may not be directly adjacent to each other (e.g., precision treatment). Also for example, implants 505a and 505b may be implanted in areas such as between knuckles 202 and/or other areas of dorsal area 205 to reduce a bony (e.g., or veiny) appearance of hand 200.

The exemplary disclosed apparatus and method may be used in any suitable application involving the treatment of the hands. For example, the exemplary disclosed apparatus and method may be used in any suitable application for the cosmetic treatment of the hands. The exemplary disclosed apparatus and method may be used in any suitable application for treating wrinkles and/or a bony and/or veiny appearance of hands due to aging. The exemplary disclosed apparatus and method may also be used in any suitable application for treating injury to hands such as, for example, from accidents or wounds (e.g., incurred by first responders, law enforcement, or military personnel in the line of duty).

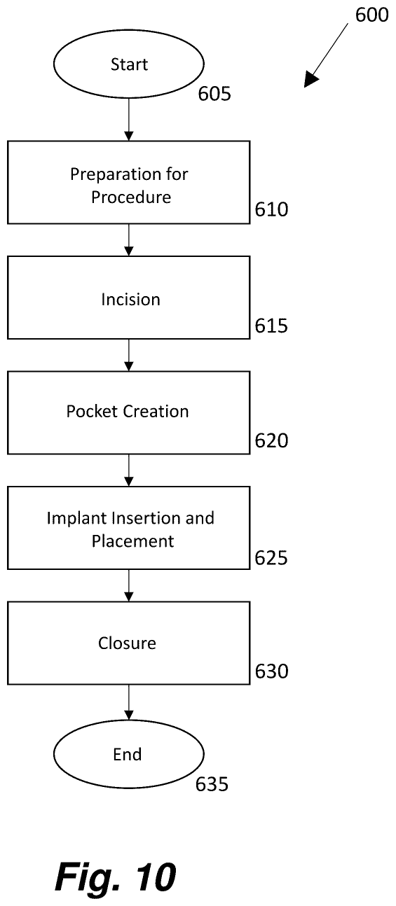

FIG. 10 illustrates an exemplary process of using the exemplary disclosed apparatus. In the exemplary disclosed process, the exemplary disclosed implant (e.g., implant 105, implant 305, implant 405, and/or implants 505a and 505b) may be inserted into a user's hand by making an incision in the skin (e.g., at dorsal area 205) of hand 200, creating a pocket beneath the dermal layer of dorsal area 205 where the exemplary disclosed implant may be placed, closing the incision (e.g., using sutures), and then allowing the patient's skin to grow around the exemplary disclosed implant to secure the implant in place.

Process 600 begins at step 605. At step 610, a user (e.g., patient) may be prepared for the implantation procedure. Hand 200 of the patient may be numbed using local anesthetic (e.g., via needle injection). Any other suitable preparation such as washing and disinfecting skin may be performed. At step 615, medical personnel (e.g., a surgeon) may make a small incision (e.g., an incision 215 for example as illustrated in FIG. 1A) through the skin to the subcutaneous layer where the exemplary disclosed implant may be placed. For example, the incision may be made to the subcutaneous layer (subcutis) of the skin.

The incision (e.g., incision 215) may be a transverse incision. For example, incision 215 may be made in a radial-ulnar direction (e.g., a side-to-side direction) across the hand at dorsal area 205 of hand 200 (e.g., perpendicular to a longitudinal axis of the hand and arm). The exemplary disclosed transverse incision may reduce or substantially minimize cosmesis. For example, incision 215 may result in scars that are less noticeable and/or relatively easier to conceal under clothing (e.g., as compared to greater scarring that may occur with vertical incisions). Incision 215 may be a low transverse incision that may be associated with relatively less post-operative pain and/or relatively faster healing (e.g., as compared to vertical incisions). Incision 215 may be made at the proximal row of carpal bones (e.g., the row closest to the forearm) at dorsal area 205. For example, the surgeon may cut the dorsal wrist crease at the proximal row using scissors to create the pocket described below. In at least some exemplary embodiments, an incision (e.g., a small incision) may be made over the dorsum (e.g., a blunt dissection parallel to the skin creases to create the exemplary disclosed pocket). Deep structures such as extensor tendons, veins (e.g., cephalic and/or basilic), and/or sensory nerves (e.g., superficial radial nerve) may be avoided while making incision 215. Incision 215 may be made using scissors and/or any other suitable surgical tool.

Returning to FIG. 10 at step 620, a pocket may be created to receive the exemplary disclosed implant (e.g., implant 105, implant 305, implant 405, and/or implants 505a and 505b). Any suitable surgical tool such as a dermal elevator may be used to gently separate the subcutis and the fascia of hand 200 at dorsal area 205, thereby creating a pocket (e.g., separating a subcutis 225 and a fascia 230 to form a pocket 220 for example as illustrated in FIG. 1D) in which the exemplary disclosed implant may be inserted. The soft tissue of dorsal area 205 may be thereby lifted away from underlying bone and other portions of hand 200, providing a space where the exemplary disclosed implant may be placed and secured. For example, the exemplary disclosed implant may be positioned within the pocket and in at least some exemplary embodiments attached or secured (e.g., to soft tissue, bone, and/or any other desired portions of hand 200, if desired). Scar tissue may develop in pocket 220 and may form around the exemplary disclosed implant, which may help to keep the implant in place. Also for example, the tissue of dorsal area 205 adjacent to the exemplary disclosed implant may respond (e.g., deform or adjust) to help hold the implant in place. The pocket may be sized and formed to fit a configuration of the exemplary disclosed implant (e.g., implant 105, implant 305, implant 405, and/or implants 505a and 505b). Returning to FIG. 10 at step 625, the exemplary disclosed implant may be inserted (e.g., carefully inserted) into the pocket created at step 620.

Pocket 220 may be any suitable size. For example, pocket 220 may be slightly smaller than a size of dorsal area 205, and may thereby vary based on a size of dorsal area 205 (e.g., based on a size of a hand of the user such as a patient). Pocket 220 may be based on a size of the patient's hand and/or a size and/or shape of the exemplary disclosed implant to be inserted. In at least some exemplary embodiments, pocket 220 may be between about 75% and about 95%, or between about 80% and about 90% of a size of dorsal area 205 in which the exemplary disclosed implant is disposed. Pocket 220 may extend to the underlying interosseous fascia and/or metacarpal periosteum.

At step 630, the incision made at step 615 may be closed. For example, the incision may be closed with sutures to close (e.g., seal) the skin of dorsal area 205 around the exemplary disclosed implant. Any suitable sutures may be used such as, for example, synthetic, absorbable, and/or monofilament surgical sutures. The exemplary disclosed sutures may be formed from any suitable material such as, for example, copolymer material (e.g., glycolide and epsilon-caprolactone). In at least some exemplary embodiments, subcutaneous running sutures may be used. In at least some exemplary embodiments, Monocryl® sutures provided by Johnson & Johnson may be used. Also for example, 5-0 nylon sutures may be used. Process 600 ends at step 635.

The exemplary disclosed implant (e.g., implant 105, implant 305, implant 405, and/or implants 505a and 505b) may be later removed from hand 200 if desired. For example, hand 200 may be prepared and an incision may be made similarly to as described above regarding steps 610 and 615. The exemplary disclosed implant may be carefully removed (e.g., via a second incision that may be made at a same location of incision 215 or at another location). The incision may then be closed similarly to as described above regarding step 630.

The exemplary disclosed process may be performed as an outpatient surgical procedure. Pain during surgery may be addressed using any suitable medication such as, for example, lidocaine and/or epinephrine. For example, local anesthesia may be used.

The exemplary disclosed incision (e.g., transverse incision such as a low transverse incision) and the exemplary disclosed implant may be suitable for use with a patient's wrist (e.g., at dorsal area 205). For example, the exemplary disclosed incision and implant may be suitable for use at the skin covering the wrist including the carpal bones (e.g., the eight bones that form the wrist, including two rows of carpal bones that connect to the two bones, the ulna bone and the radius bone, of the arm). The skin that covers the dorsum (e.g., at dorsal area 205) of the hand differs from the skin covering the palm, with the skin of the dorsum of the hand (e.g., at dorsal area 205) being relatively thin and pliable and being attached to bones of the hand via loose areolar tissue (e.g., at which veins and lymphatics are disposed). The exemplary disclosed incision and implant may be suitable for use at this area (e.g., at dorsal area 205), which may be subject to conditions such as edema, skin avulsion damage, and local flap creation. The dorsal skin of the hand generally bears hair over the proximal phalanges and the ulnar aspect. The exemplary disclosed incision and implant may be suitable for counteracting natural loss of subcutaneous fat, elastin, and collagen with age (e.g., and accompanying thinning of the skin that makes tendons and veins more visible, which can be worsened by sun exposure).

The invention includes other illustrative embodiments (“Embodiments”) as follows.

Embodiment 1: A method, comprising: making an incision in a dorsal area of a hand; creating a pocket at the incision in the dorsal area by separating a subcutis and a fascia of the hand; and inserting a subdermal hand implant in the pocket; wherein the subdermal hand implant includes a sheet of malleable material.

Embodiment 2: The method of Embodiment 1, wherein the subdermal hand implant includes a plurality of separate sheets of malleable material; and one of the plurality of separate sheets is a triangular-shaped or heart-shaped separate sheet configured to be disposed between an index finger and a thumb of the dorsal area.

Embodiment 3: The method of Embodiment 1, wherein the subdermal hand implant is a permanent-form implant that is non-dissipating and non-temporary.

Embodiment 4: The method of Embodiment 1, further comprising removing the subdermal hand implant from the pocket.

Embodiment 5: The method of Embodiment 1, further comprising: closing the incision; and later making a second incision and removing the subdermal hand implant from the pocket through the second incision; wherein the incision is a transverse incision.

Embodiment 6: The method of Embodiment 1, wherein inserting the subdermal hand implant in the pocket includes covering a plurality of dorsal veins of the hand.

Embodiment 7: The method of Embodiment 1, wherein the sheet of malleable material is configured in a dorsal hand shape that is a contoured shape corresponding to a shape of a wrist.

Embodiment 8: The method of Embodiment 1, wherein each of a length dimension and a width dimension of the sheet of malleable material is between 5 times and 50 times greater than a thickness dimension of the sheet of malleable material.

Embodiment 9: The method of Embodiment 1, wherein the sheet of malleable material includes one or more thick portions having a thickness greater than a remainder of the sheet of malleable material that has a constant thickness.

Embodiment 10: The method of Embodiment 9, wherein the one or more thick portions are disposed between knuckles of the hand when the subdermal hand implant is disposed in the pocket.

Embodiment 11: A subdermal hand implant configured to be implanted in a dorsal area of a hand, comprising: a sheet of malleable material; wherein the sheet of malleable material is a permanent-form implant that is non-dissipating and non-temporary; wherein the sheet of malleable material is configured in a dorsal hand shape; and wherein each of a length dimension and a width dimension of the sheet of malleable material is between 5 times and 50 times greater than a thickness dimension of the sheet of malleable material.

Embodiment 12: The subdermal hand implant of Embodiment 11, wherein the sheet of malleable material includes one or more thick portions having a thickness greater than a remainder of the sheet of malleable material that has a constant thickness.

Embodiment 13: The subdermal hand implant of Embodiment 11, wherein the sheet of malleable material is formed from silicone gel or from a silicone pouch filled with fat.

Embodiment 14: The subdermal hand implant of Embodiment 11, wherein the sheet of malleable material includes a malleable shell housing a fluid fill.

Embodiment 15: A method, comprising: making a transverse incision in a dorsal area of a hand at a proximal row of carpal bones; creating a pocket at the transverse incision in the dorsal area by separating a subcutis and a fascia of the hand; inserting a subdermal hand implant in the pocket; closing the transverse incision; and making a second incision and removing the subdermal hand implant from the pocket through the second incision; wherein the subdermal hand implant includes a sheet of malleable material; and wherein the sheet of malleable material is a permanent-form sheet that is non-dissipating and non-temporary.

Embodiment 16: The method of Embodiment 15, wherein inserting the subdermal hand implant in the pocket includes covering a plurality of dorsal veins of the hand.

Embodiment 17: The method of Embodiment 15, wherein the sheet of malleable material is configured in a dorsal hand shape.

Embodiment 18: The method of Embodiment 15, wherein each of a length dimension and a width dimension of the sheet of malleable material is between 10 times and 40 times greater than a thickness dimension of the sheet of malleable material.

Embodiment 19: The method of Embodiment 15, wherein the sheet of malleable material includes one or more thick portions having a thickness greater than a remainder of the sheet of malleable material that has a constant thickness.

Embodiment 20: The method of Embodiment 19, wherein the one or more thick portions are disposed between knuckles of the hands when the subdermal hand implant is disposed in the pocket.

In at least some exemplary embodiments, the exemplary disclosed apparatus and method may provide an efficient and effective technique for cosmetically treating hands. For example, the exemplary disclosed apparatus and method may provide for an efficient and effective technique for addressing wrinkles, a bony appearance of older hands, and/or a veiny appearance of older hands. The exemplary disclosed apparatus and method may also provide for an efficient and effective technique for addressing injury to hands.

It will be apparent to those skilled in the art that various modifications and variations can be made to the exemplary disclosed system, apparatus, and method. Other embodiments will be apparent to those skilled in the art from consideration of the specification and practice of the exemplary disclosed apparatus, system, and method. It is intended that the specification and examples be considered as exemplary, with a true scope being indicated by the following claims.

What is claimed is:

1. A method, comprising:
making an incision in a dorsal area of a hand;
creating a pocket at the incision in the dorsal area by separating a subcutis and a fascia of the hand; and
inserting a subdermal hand implant in the pocket;
wherein the subdermal hand implant includes a sheet of malleable material.

2. The method of claim 1, wherein:
the subdermal hand implant includes a plurality of separate sheets of malleable material; and
one of the plurality of separate sheets is a triangular-shaped or heart-shaped separate sheet configured to be disposed between an index finger and a thumb of the dorsal area.

3. The method of claim 1, wherein the subdermal hand implant is a permanent-form implant that is non-dissipating and non-temporary.

4. The method of claim 1, further comprising removing the subdermal hand implant from the pocket.

5. The method of claim 1, further comprising:
closing the incision; and
later making a second incision and removing the subdermal hand implant from the pocket through the second incision;
wherein the incision is a transverse incision.

6. The method of claim 1, wherein inserting the subdermal hand implant in the pocket includes covering a plurality of dorsal veins of the hand.

7. The method of claim 1, wherein the sheet of malleable material is configured in a dorsal hand shape that is a contoured shape corresponding to a shape of a wrist.

8. The method of claim 1, wherein each of a length dimension and a width dimension of the sheet of malleable material is between 5 times and 50 times greater than a thickness dimension of the sheet of malleable material.

9. The method of claim 1, wherein the sheet of malleable material includes one or more thick portions having a thickness greater than a remainder of the sheet of malleable material that has a constant thickness.

10. The method of claim 9, wherein the one or more thick portions are disposed between knuckles of the hand when the subdermal hand implant is disposed in the pocket.

11. A subdermal hand implant configured to be implanted in a dorsal area of a hand, comprising:
a sheet of malleable material;
wherein the sheet of malleable material is a permanent-form implant that is non-dissipating and non-temporary;
wherein the sheet of malleable material is configured in a dorsal hand shape; and
wherein each of a length dimension and a width dimension of the sheet of malleable material is between 5 times and 50 times greater than a thickness dimension of the sheet of malleable material.

12. The subdermal hand implant of claim 11, wherein the sheet of malleable material includes one or more thick portions having a thickness greater than a remainder of the sheet of malleable material that has a constant thickness.

13. The subdermal hand implant of claim 11, wherein the sheet of malleable material is formed from silicone gel or from a silicone pouch filled with fat.

14. The subdermal hand implant of claim 11, wherein the sheet of malleable material includes a malleable shell housing a fluid fill.

15. A method, comprising:
making a transverse incision in a dorsal area of a hand at a proximal row of carpal bones;
creating a pocket at the transverse incision in the dorsal area by separating a subcutis and a fascia of the hand;
inserting a subdermal hand implant in the pocket;
closing the transverse incision; and
making a second incision and removing the subdermal hand implant from the pocket through the second incision;
wherein the subdermal hand implant includes a sheet of malleable material; and
wherein the sheet of malleable material is a permanent-form sheet that is non-dissipating and non-temporary.

16. The method of claim 15, wherein inserting the subdermal hand implant in the pocket includes covering a plurality of dorsal veins of the hand.

17. The method of claim 15, wherein the sheet of malleable material is configured in a dorsal hand shape.

18. The method of claim 15, wherein each of a length dimension and a width dimension of the sheet of malleable material is between 10 times and 40 times greater than a thickness dimension of the sheet of malleable material.

19. The method of claim 15, wherein the sheet of malleable material includes one or more thick portions having a thickness greater than a remainder of the sheet of malleable material that has a constant thickness.

20. The method of claim 19, wherein the one or more thick portions are disposed between knuckles of the hands when the subdermal hand implant is disposed in the pocket.
